(19) United States
(12) Patent Application Publication
(10) Pub. No.: US 2015/0258004 A1
(43) Pub. Date: Sep. 17, 2015

(54) PROCESS FOR COSMETIC TREATMENT OF THE HAIR USING A COMPOSITION COMPRISING A DISPERSION OF PARTICLES COMPRISING A MATERIAL CAPABLE OF CHANGING STATE UNDER THE EFFECT OF A STIMULUS

(71) Applicant: L'OREAL, Paris (FR)

(72) Inventors: Gregory Plos, Paris (FR); Henri Samain, Bievres (FR); Laetitia Feuillette, Paris (FR); Christophe Jacques, Herblay (FR); Bradford Pistorio, Chicago, IL (US)

(21) Appl. No.: 14/435,775

(22) PCT Filed: Oct. 15, 2013
(86) PCT No.: PCT/EP2013/071507

§ 371 Date: Apr. 15, 2015

Related U.S. Application Data
(60) Provisional application No. 61/737,905, filed on Dec. 17, 2012.

(30) Foreign Application Priority Data
Oct. 15, 2012 (FR) 1259794

(51) Int. Cl. A61K 8/81; A45D 7/02; A61Q 5/06; A61K 8/02; A61K 8/04

(52) U.S. Cl. CPC A61K 8/8111; A61K 8/0241; A61K 8/042; A61K 8/04; A61Q 5/06; A45D 7/02; A61K 2800/48; A61K 2800/654

(57) ABSTRACT

The present invention relates to a process for cosmetic treatment of the hair, in particular a hair shaping process, comprising the following steps: applying to the hair a composition comprising a liquid solvent system and a dispersion of particles that are insoluble in the solvent system, the particles comprising a material capable of changing state under the effect of an external stimulus, before, during or after the applying of the composition, shaping the hair to the desired hairstyle, then or simultaneously, applying one or more stimuli enabling the change of state of the material, stopping the stimulus or stimuli for obtaining fixing of the hairstyle.

The invention relates to a process for cosmetic treatment of the hair, in particular for shaping and/or form retention of the hairstyle, using a composition comprising a dispersion of particles of material capable of changing state under the effect of a stimulus.

The hair products for shaping and/or form retention of the hairstyle that are the most widespread on the cosmetics market are spray compositions, such as lacquers and sprays. They are essentially constituted of an alcoholic or aqueous solution and one or more materials, generally polymer resins, also known as fixing components, of which the function is to form welds between the hairs, as a mixture with various cosmetic adjuvants. Lacquers make it possible in particular to obtain effective and barely perceptible spot fixing. Sprays and lacquers are generally dispensed by means of an aerosol device or a pump, thereby generating additional waste compared with a simple bottle, a tube or a jar.

There are, moreover, styling methods which use heat treatment to shape the hairstyle. Document FR 2 811 886 describes, for example, a styling process consisting in applying to the hair a composition containing a fixing polymer and a hot-melt polymer and in heating at a temperature close to or above the melting point of the polymer, and then in allowing to cool. Since the hot-melt polymer is combined with a fixing polymer and is soluble in the formulating medium, fixing of the hairstyle is obtained by coating of the hair, which is relatively perceptible.

Document DE 2 810 130 describes a process for shaping the head of hair which consists of the application, to the head of hair, of a powder, a mesh or a net of polymer, followed by the application of heat to melt the polymer and thus to solidify the hairstyle. The polymer softens or melts at a temperature between 35° C. and 120° C., and heat is supplied at a temperature between 40° C. and 150° C. with tools such as a hood or “radiant heating”. However, powders, meshes or nets are relatively difficult to apply to the hair with precision.

There is a need to obtain barely perceptible effective fixing of the hairstyle without the need to use a particular dispensing device, with a simple and precise application movement.

The applicant has found that it is possible to remedy the drawbacks of the prior art and to meet the abovementioned objectives.

The subject of the present invention is a process for cosmetic treatment of the hair, in particular a hair shaping process, comprising the following steps:
applying to the hair a composition comprising a liquid solvent system and a dispersion of particles that are insoluble in the solvent system, the particles comprising at least one material capable of changing state under the effect of an external stimulus,
before, during or after the applying of the composition, shaping the hair to the desired hairstyle, then or simultaneously,
applying one or more stimuli enabling the change of state of the material,
stopping the stimulus or stimuli for obtaining fixing of the hairstyle.

During the application of the stimulus or stimuli, the material changes state, going in particular from a solid state to a liquid state. The particles melt and form spot welds between the hairs. The fixing is comparable to the type of fixing obtained with a lacquer without having recourse to the use of an aerosol and is much less perceptible than with a gel.

Other subjects, characteristics, aspects and advantages of the invention will become even more clearly apparent on reading the description and examples that follow.

The expression “at least one” is equivalent to the expression “one or more”.

The stimulus enabling the change of state of the material is chosen from heat, mechanical, pH-variation and light stimuli.

Preferably, the stimulus enabling the change of state of the material is a heat stimulus.

The heat stimulus is applied by means of a hairdryer, a heating hood, infrared radiation or microwaves.

Other stimuli may be envisaged: a mechanical stimulus can be generated by friction, by pressure or by vibration, the pH variation can be generated by precipitation of a compound; light, in particular visible or UV light, can be applied by means of a lamp or by photoreaction.

The composition applied in the process according to the invention comprises a solvent system and a dispersion of particles that are insoluble in the solvent system, the particles that are insoluble in the solvent comprising a material capable of changing state.

The term “solvent system” is intended to mean an isolated solvent or a mixture of solvents.

For the purposes of the present invention, the expression “particle that is insoluble in a solvent system” is intended to mean a particle of which the solubility in the solvent system at 25° C. and at atmospheric pressure is less than 0.1%, better still less than 0.001% and which is present in the solvent in solid or pasty form, preferably solid form.

According to one embodiment, the particles that are insoluble in the solvent system comprise at least one material of which the softening or melting point is between 30 and 150° C., limits inclusive. Preferably, the compound has a softening or melting point between 30 and 120° C., even more preferentially between 35 and 80° C., limits inclusive.

The softening or melting point values can be determined by the DSC (Differential Scanning calorimetry) method; the softening or melting point then corresponds to the melting peak and the temperature rise is 5 or 10° C./min. The melting point can also be measured using a Kofler bench.

The particles that are insoluble in the solvent may be particles of thermoplastic polymers, waxes or pasty fatty substances, or of a mixture of these compounds.

Preferably, the particles that are insoluble in the solvent system do not contain silicon atoms.

The thermoplastic polymers may be chosen from homopolymers or copolymers, in particular block and/or random homopolymers or copolymers, such as polyacrylics, polymethacrylics, for instance polyacrylamides or polymethacrylic acids, polyolefins such as polypropylenes and polyethylenes, polystyrenes, poly(vinyl halides) such as PVP (polyvinyl chloride), poly(vinyl nitriles), polyurethanes, polyesters, polyvinyls, polyvinyl esters, polycarbonates, polysulfones, polysulfonamides, polycyclics which have a ring in the main chain, such as polyphenylenes, polymers which have a heteroatom in the main chain, such as polyamides, polyoxyphenylenes.

In the case of the homopolymers, they may be more particularly chosen from homopolymers constituted of the following monomers:

monomers of which the homopolymers have a glass transition temperature of greater than or equal to 40° C., chosen, preferably, from the following monomers:

methacrylates of formula CH₂═C(CH₃) COOR1

in which R1 represents a linear or branched alkyl group containing from 1 to 4 carbon atoms, such as a methyl, ethyl, propyl or isobutyl group, it being possible for said alkyl group to also be optionally substituted with one or more substituents chosen from hydroxyl groups and halogen atoms (CI, Br, I, F), or R1 represents a C₄ to C₁₂ cycloalkyl group.

acrylates of formula CH₂═CH COOR2

in which R2 represents a C₄ to C₁₂ cycloalkyl group such as isobornyl acrylate or a tert-butyl group,

(meth)acrylamides of formula:

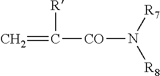

in which R7 and R8, which may be identical or different, each represent a hydrogen atom or a linear or branched C₁ to C₁₂ alkyl group such as an n-butyl, t-butyl, isopropyl, isohexyl, isooctyl or isononyl group; or R7 represents H and R8 represents a 1,1-dimethyl-3-oxobutyl group,
and R′ denotes H or methyl. Examples of monomers that may be mentioned include N-butylacrylamide, N-t-butylacrylamide, N-isopropylacrylamide, N,N-dimethylacrylamide and N,N-dibutylacrylamide,

styrene and derivatives thereof such as chlorostyrene,

and mixtures thereof.

Monomers that are particularly preferred are methyl methacrylate, isobutyl methacrylate and isobornyl (meth)acrylate, trifluoroethyl methacrylate, styrene and mixtures thereof.

Preferably, the thermoplastic polymers are chosen from polyolefins such as polyethylenes and polypropylenes, polyamides, polyethers, polyurethanes or copolymers of these compounds.

The composition may also comprise wax particles.

The term “wax” is intended to mean a lipophilic compound which is solid at ambient temperature (25° C.), with a reversible solid/liquid change of state, which has a melting point of greater than or equal to 30° C., which may be up to 200° C.

For the purposes of the invention, the melting point corresponds to the temperature of the most endothermic peak observed in thermal analysis (DSC) as described in the standard ISO 11357-3; 1999. The melting point of the wax may be measured using a differential scanning calorimeter (DSC), for example the calorimeter sold under the name MDSC 2920 by the company TA Instruments.

The measurement protocol is as follows:

A sample of 5 mg of wax placed in a crucible is subjected to a first temperature rise ranging from −20° C. to 100° C., at a heating rate of 10° C./minute, it is then cooled from 100° C. to −20° C. at a cooling rate of 10° C./minute and finally it is subjected to a second temperature rise ranging from −20° C. to 100° C., at a heating rate of 5° C./minute. During the second temperature rise, the variation in the difference in power absorbed by the empty crucible and by the crucible containing the sample of wax is measured as a function of the temperature. The melting point of the compound is the temperature value corresponding to the top of the peak of the curve representing the variation in the difference in power absorbed as a function of the temperature.

The wax may be chosen from waxes of natural origin, synthetic waxes, which are solid at ambient temperature, and mixtures thereof.

For the purposes of the present invention, a “wax of natural origin” implies that the wax is not a synthetic wax, i.e. is not obtained by chemical synthesis.

It is derived from a vegetable, animal or mineral source.

This natural wax may, however, have undergone secondary treatments of refining and/or purification type, or else a chemical reaction, such as esterification or etherification, for example. These modified waxes of natural origin are also suitable for the present invention.

Generally, a wax taken into consideration in the context of the present invention is a lipophilic compound which is solid at ambient temperature (25° C.).

By way of illustration of waxes of natural origin that are suitable for the invention, mention may be made in particular of hydrocarbon-based waxes, for instance beeswax, lanolin wax, Chinese insect waxes, rice bran wax, carnauba wax, candelilla wax, ouricury wax, esparto wax, berry wax, shellac wax, Japan wax, sumac wax, montan wax, orange wax and lemon wax, cork fibre wax, sugarcane wax, soya bean wax, bayberry wax, retamo wax, palm kernel wax, palm wax, whale wax, Astrocaryum murumuru butter, Myrica pubescen wax, oxyethylenated mango oil and derived products thereof.

By way of illustration of derived products, mention may in particular be made of modified waxes of natural origin, for instance the polyethylene glycol-8 modified beeswax sold under the trade name “Apifil” by the company Gattefosse France, the polyglyceryl-3 modified beeswax sold under the trade name “Cera Belling®” by the company Koster Keunen, oxypropylenated lanolin wax, and mixtures thereof.

Mention may also be made of waxes obtained by catalytic hydrogenation of animal or vegetable oils containing linear or branched C₈-C₃₂ fatty chains. Among these, mention may be made in particular of isomerized jojoba oil such as the trans-isomerized partially hydrogenated jojoba oil manufactured or sold by the company Desert Whale under the trade reference Iso-Jojoba-50®, hydrogenated sunflower oil, hydrogenated castor oil, hydrogenated coconut oil and hydrogenated lanolin oil.

Beeswax will preferably be used.

The synthetic waxes may be chosen from nonpolar waxes and ester waxes.

The term “ester wax” is intended to mean, according to the invention, a wax comprising at least one ester function.

The waxes obtained by hydrogenation of castor oil esterified with cetyl alcohol, sold under the names Phytowax ricin 16L64® and 22L73® by the company Sophim, may also be used as ester wax.

As ester wax that may be used, mention may also be made of the bis(1,1,1-trimethylolpropane) tetrastearate sold under the name Hest 2T-4S® by the company Heterene.

The term “ester wax” is also intended to mean esters of formula R₁COOR₂ in which R₁ and R₂ represent linear, branched or cyclic aliphatic chains of which the number of atoms ranges from 10 to 50, which may contain a heteroatom such as 0, N or P and the melting point of which ranges from 25 to 120° C. Mention will, for example, be made of cetyl palmitate, hydroxyoctacosanol hydrostearate, bis(trimethylolpropane) tetralaurate, and the mixture of dilauryl adipate and di(tetradecyl) adipate. The nonpolar waxes are in particular hydrocarbon-based waxes constituted solely of carbon and hydrogen atoms and devoid of heteroatoms, such as N, O, Si and P.

By way of illustration of the nonpolar waxes that are suitable for the invention, mention may in particular be made of hydrocarbon-based waxes, such as microcrystalline waxes, paraffin waxes, ozokerite, ceresin and polyethylene waxes, waxes obtained by Fisher-Tropsch synthesis and waxy copolymers and also esters thereof.

Mention may be made, as ozokerite, of Ozokerite Wax SP 1020 P.

As microcrystalline waxes that may be used, mention may be made of Multiwax W 445® sold by the company Sonneborn, and Microwax HW® and Base Wax 30540® sold by the company Paramelt.

Mention may also be made of silicone waxes, such as alkyl dimethicones or alkoxy dimethicones containing a C₁₀-C₄₅ alkyl group, poly(di)methylsiloxane esters which are solid at 30° C. and the ester chain of which comprises at least 10 carbon atoms, fluoropolymethyldimethysiloxanes, and mixtures thereof.

The use of linear C₁₀-C₃₀ fatty alcohols, for example stearyl alcohol, cetyl alcohol, lauryl alcohol, myristyl alcohol or docosanol, may also be envisaged.

Preferably, the bis(1,1,1-trimethylolpropane) tetrastearate sold under the name Hest 2T-45® by the company Heterene will be used.

The material capable of changing state may also comprise at least one pasty fatty substance.

For the purposes of the present invention, the term “pasty fatty substance” is intended to mean a lipophilic fatty compound with a reversible solid/liquid change of state, comprising at a temperature of 23° C. a liquid fraction and a solid fraction.

In other words, the starting melting point of the pasty compound can be less than 23° C. The liquid fraction of the pasty compound, measured at 23° C., can represent from 9% to 97% by weight of the compound. This liquid fraction at 23° C. preferably represents between 15% and 85% by weight and more preferably between 40% and 85% by weight.

Preferably, the pasty fatty substances exhibit an end melting point of less than 60° C.

Preferably, the pasty fatty substances exhibit a hardness of less than or equal to 6 MPa.

Preferably, the pasty fatty substances have, in the solid state, an anisotropic crystal organization, which is visible by X-ray observation.

For the purposes of the invention, the melting point corresponds to the temperature of the most endothermic peak observed in thermal analysis (DSC) as described in the standard ISO 11357-3; 1999. The melting point of a pasty substance or of a wax can be measured using a differential scanning calorimeter (DSC), for example the calorimeter sold under the name DSC Q2000 by TA Instruments.

As regards the measurement of the melting point and the determination of the end melting point, the sample preparation and measurement protocols are as follows:

A sample of 5 mg of pasty fatty substance, preheated to 80° C. and withdrawn with magnetic stirring using a spatula which is also heated, is placed in a hermetic aluminium capsule, or crucible. Two tests are carried out to ensure the reproducibility of the results.

The measurements are carried out on the abovementioned calorimeter. The oven is flushed with nitrogen. Cooling is carried out with an RCS 90 heat exchanger. The sample is subsequently subjected to the following protocol: it is first of all placed at a temperature of 20° C., is then subjected to a first temperature rise passing from 20° C. to 80° C. at a heating rate of 5° C./minute, is then cooled from 80° C. to −80° C. at a cooling rate of 5° C./minute and, finally, is subjected to a second temperature rise passing from −80° C. to 80° C. at a heating rate of 5° C./minute. During the second temperature rise, the variation in the difference in power absorbed by the empty crucible and by the crucible containing the sample of pasty substance or wax is measured as a function of the temperature. The melting point of the compound is the temperature value corresponding to the top of the peak of the curve representing the variation in the difference in power absorbed as a function of the temperature.

The end melting point corresponds to the temperature at which 95% of the sample has melted.

The liquid fraction by weight of the pasty compound at 23° C. is equal to the ratio of the enthalpy of fusion consumed at 23° C. to the enthalpy of fusion of the pasty compound.

The enthalpy of fusion of the pasty compound is the enthalpy consumed by the compound in order to pass from the solid state to the liquid state. The pasty compound is said to be in the solid state when all of its mass is in the crystalline solid form. The pasty compound is said to be in the liquid state when all of its mass is in the liquid form.

The enthalpy of fusion of the pasty compound is equal to the integral of the entire melting curve obtained using the abovementioned calorimeter, with a temperature rise of 5 or 10° C. per minute, according to the standard ISO 11357-3:1999. The enthalpy of fusion of the pasty compound is the amount of energy necessary to make the compound change from the solid state to the liquid state. It is expressed in J/g.

The enthalpy of fusion consumed at 23° C. is the amount of energy absorbed by the sample to change from the solid state to the state which it exhibits at 23° C., consisting of a liquid fraction and a solid fraction.

The liquid fraction of the pasty compound measured at 32° C. preferably represents from 30% to 100% by weight of the compound, preferably from 50% to 100% by weight and more preferably from 60% to 100% by weight of the compound. When the liquid fraction of the pasty compound, measured at 32° C., is equal to 100%, the temperature of the end of the melting range of the pasty compound is less than or equal to 32° C.

The liquid fraction of the pasty compound measured at 32° C. is equal to the ratio of the enthalpy of fusion consumed at 32° C. to the enthalpy of fusion of the pasty compound. The enthalpy of fusion consumed at 32° C. is calculated in the same way as the enthalpy of fusion consumed at 23° C.

As regards the measurement of the hardness, the sample preparation and measurement protocols are as follows:

The pasty fatty substance is placed in a mould with a diameter of 75 mm which is filled to approximately 75% of its height. In order to overcome the thermal history and to control the crystallization, the mould is placed in a Vötsch VC0018 programmable oven, where it is first of all placed at a temperature of 80° C. for 60 minutes, then cooled from 80° C. to 0° C. at a cooling rate of 5° C./minute, then left at the stabilized temperature of 0° C. for 60 minutes, then subjected to a temperature rise passing from 0° C. to 20° C. at a heating rate of 5° C./minute and then left at the stabilized temperature of 20° C. for 180 minutes.

The compressive force measurement is carried out with the TA/TX2i texture analyser from Swantech. The spindle used is chosen according to the texture:
cylindrical steel spindle with a diameter of 2 mm, for very rigid starting materials;
cylindrical steel spindle with a diameter of 12 mm, for relatively non-rigid starting materials.

The measurement comprises 3 stages: a first stage after automatic detection of the surface of the sample where the spindle is displaced at the measurement rate of 0.1 mm/s and penetrates the pasty fatty substance to a penetration depth of 0.3 mm—the software records the value of the maximum force achieved; a second “relaxation” stage where the spindle remains at this position for one second and where the force is recorded after 1 second of relaxation; finally, a third “withdrawal” stage where the spindle returns to its initial position at the rate of 1 mm/s and the probe withdrawal energy (negative force) is recorded.

The hardness value measured during the first step corresponds to the maximum compression force measured in newtons divided by the area of the texturometer cylinder expressed in mm² in contact with the pasty fatty substance. The hardness value obtained is expressed in megapascals or MPa.

The pasty fatty substance is preferably chosen from synthetic fatty substances and fatty substances of vegetable origin. A pasty fatty substance can be obtained by synthesis from starting materials of vegetable origin.

The pasty fatty substance is advantageously chosen from:
lanolin and derivatives thereof,
polyol ethers chosen from pentaerythrityl ethers of a polyalkylene glycol, fatty alkyl ethers of a sugar, and mixtures thereof, the pentaerythrityl ether of polyethylene glycol comprising 5 oxyethylene units (5 OE) (CTFA name: PEG-5 Pentaerythrityl Ether), the pentaerythrityl ether of polypropylene glycol comprising 5 oxypropylene (5 OP) units (CTFA name: PPG-5 Pentaerythrityl Ether), and mixtures thereof, and more especially the PEG-5 Pentaerythrityl Ether, PPG-5 Pentaerythrityl Ether and soya bean oil mixture, sold under the name Lanolide by the company Vevy, in which mixture the constituents are in a 46/46/8 ratio by weight: 46% PEG-5 Pentaerythrityl Ether, 46% PPG-5 Pentaerythrityl Ether and 8% soya bean oil,
polymeric or non-polymeric silicone compounds,
polymeric or non-polymeric fluoro compounds,
vinyl polymers, in particular:
   olefin homopolymers and copolymers,
   hydrogenated diene homopolymers and copolymers,
   linear or branched oligomers, homopolymers or copolymers of alkyl (meth)acrylates preferably containing a C₈-C₃₀ alkyl group,
   homopolymeric and copolymeric oligomers of vinyl esters containing C₈-C₃₀ alkyl groups,
   homopolymeric and copolymeric oligomers of vinyl ethers containing C₈-C₃₀ alkyl groups,
liposoluble polyethers resulting from the polyetherification between one or more C₂-C₁₀₀ and preferably C₂-C₅₀ diols,
esters,
and/or mixtures thereof.

Among the liposoluble polyethers that are particularly preferred are copolymers of ethylene oxide and/or of propylene oxide with C₆-C₃₀ long-chain alkylene oxides, more preferably such that the weight ratio of the ethylene oxide and/or of the propylene oxide to the alkylene oxides in the copolymer is from 5:95 to 70:30. In this family, mention will in particular be made of copolymers such that the long-chain alkylene oxides are arranged in blocks having an average molecular weight from 1000 to 10000, for example a polyoxyethylene/polydodecyl glycol block copolymer such as the ethers of dodecanediol (22 mol) and of polyethylene glycol (45 OE) sold under the brand name Elfacos ST9 by Akzo Nobel.

Preference is given in particular, among the esters, to:
esters of a glycerol oligomer, in particular diglycerol esters, in particular condensates of adipic acid and of glycerol, for which a portion of the hydroxyl groups of the glycerols has reacted with a mixture of fatty acids, such as stearic acid, capric acid, isostearic acid and 12-hydroxystearic acid, such as in particular those sold under the brand name Softisan 649 by Sasol,
arachidyl propionate, sold under the brand name Waxenol 801 by Alzo,
phytosterol esters,
fatty acid triglycerides and derivatives thereof,
pentaerythritol esters,
esters of a diol dimer and of a diacid dimer, where appropriate esterified on their free alcohol or acid function(s) with acid or alcohol radicals, in particular dimer dilinoleate esters; such esters can be chosen in particular from esters with the following INCI nomenclature: hydrogenated castor oil dimer dilinoleate known under the name Risocast DA-L or also Risocast DA-H, distributed by the company Kokyu Alcohol Kogyo, bis-behenyl/isostearyl/phytosteryl dimer dilinoleyl dimer dilinoleate (Plandool G), phytosteryl isostearyl dimer dilinoleate (Lusplan PI-DA, Lusplan PHY/IS-DA), phytosteryl/isosteryl/cetyl/stearyl/behenyl dimer dilinoleate (Plandool H or Plandool S), and mixtures thereof,
mango butter, such as the product sold under the reference Lipex 203 by the company AarhusKarlshamn,
hydrogenated soya bean oil, hydrogenated coconut oil, hydrogenated rapeseed oil or mixtures of hydrogenated vegetable oils, such as the soya bean, coconut, palm and rapeseed hydrogenated vegetable oil mixture, for example the mixture sold under the reference Akogel® by the company AarhusKarlshamn (INCI name: Hydrogenated Vegetable Oil),
shea butter, in particular that having the INCI name Butyrospermum Parkii Butter, such as that sold under the reference Sheasoft® by AarhusKarlshamn,
cocoa butter, in particular the product which is sold under the name CT Cocoa
Butter Deodorized by the company Dutch Cocoa BV or the product which is sold under the name Beurre De Cacao NCB HD703 758 by the company Barry Callebaut,
shorea butter, in particular the product which is sold under the name Dub Shorea T by the company Stearinerie Dubois,
and mixtures thereof.

More particularly, the pasty fatty substance is chosen from esters of a diol dimer and a diacid dimer, where appropriate esterified on their free alcohol or acid function(s) with acid or alcohol radicals, in particular dimer dilinoleate esters; mango butter, hydrogenated soya bean oil, hydrogenated coconut oil, hydrogenated rapeseed oil, mixtures of hydrogenated vegetable oils, such as the soya bean, coconut, palm and rapeseed hydrogenated vegetable oil mixture, shea butter, cocoa butter, shorea butert, and mixtures thereof.

According to one preferred embodiment, the pasty fatty substance is chosen from a mixture of hydrogenated soya bean, coconut, palm and rapeseed vegetable oils, shea butter, cocoa butter, shorea butter, and mixtures thereof, and more particularly those referenced above.

According to another embodiment, the particles that are insoluble in the solvent system comprise at least one material capable of changing state in response to a change in pH, in particular around a pH of 8 or 9.

Mention will, for example, be made of ionic polymers, in particular anionic polymers, such as the polymers sold under the names Resyn 28 2930 by the company Akzo Nobel and Gantrez ES 425 L by the company ISP.

Preferably, the particles that are insoluble in the solvent system comprise at least one material capable of reversibly changing state. Thus, when the stimulus is stopped, the material returns to its initial state.

Advantageously, the particle(s) has (have) a number-average primary size ranging from 0.01 to 500 μm, preferably from 0.1 to 300 μm and more preferentially from 1 to 250 μm. According to a preferred embodiment, the particle(s) has (have) a number-average primary size ranging from 1 to 500 μm.

For the purposes of the present invention, the term “primary particle size” is intended to mean the maximum dimension that it is possible to measure between two diametrically opposed points of an individual particle.

The size of the particles may be determined by transmission electron microscopy or by measuring the specific surface area by the BET method or using a laser particle sizer.

The particles that are insoluble in the solvent may have various shapes, for example sphere-shaped, glitter flakes, needles or platelets and preferably they are substantially spherical.

The particles that are insoluble in the solvent are prepared by grinding the material of which they are constituted in a knife mill and then sieving the particles obtained. The fraction between 0.01 μm and 500 μm is harvested and dispersed in the formulation medium.

In the case of mixtures of several materials, the materials are premixed in the molten phase.

Preferably, the particles are present in the composition applied in the process according to the invention in proportions of at least 0.05% by weight, preferably ranging from 0.1% to 95% by weight, more preferentially from 1% to 50% by weight and better still from 2% to 30% by weight, relative to the total weight of the composition.

The composition applied in the process according to the invention comprises a solvent system.

As indicated previously, the solvent system comprises one or more solvents.

Preferably, the solvent(s) has (have) a boiling point above the melting/softening point of the material capable of changing state.

The composition comprises an aqueous, alcoholic or aqueous-alcoholic solvent, preferably an aqueous-alcoholic solvent.

Preferably, the composition comprises water, preferably in a content of greater than or equal to 5% by weight relative to the total weight of the composition. The water content preferentially ranges from 5% to 98%, preferably from 30% to 95%, better still from 50% to 90% and even better still from 70% to 90% by weight relative to the total weight of the composition.

The composition may also comprise one or more water-soluble liquid organic solvents preferably chosen from monoalcohols such as ethanol or isopropanol; polyols such as propylene glycol, butylene glycol or glycerol; polyol ethers; and mixtures thereof. The organic solvent is preferably ethanol.

Preferably, the composition does not comprise any fixing polymer. The term “fixing polymer” is intended to mean a polymer capable of conferring a shape on the head of hair without the application of a stimulus.

The composition according to the invention may also comprise one or more thickeners different from the particles of the invention which may be chosen from natural or synthetic, associative or non-associative polymeric thickeners, and non-polymeric thickeners, in particular inorganic thickeners.

The term “thickener” is intended to mean a compound which, by virtue of its presence, makes it possible to increase the viscosity of the composition by at least 100 cps at 25° C. and at a shear rate of 1 s⁻¹.

Examples of polymeric thickeners that may be mentioned include cellulose thickeners, for example hydroxyethylcellulose, hydroxypropylcellulose and carboxymethylcellulose, guar gum and its derivatives, for example hydroxypropyl guar, sold by the company Rhodia under the reference Jaguar HP 105, gums of microbial origin, such as xanthan gum and scleroglucan gum, carrageenan, for example the carrageenan powder sold by the company Cargill under the reference Satiagum UTC 30, synthetic polymeric thickeners, resulting from radical polymerization reactions or polycondensation reactions such as crosslinked homopolymers of acrylic acid or of acrylamidopropanesulfonic acid, for example carbomers, or non-ionic, anionic or amphoteric associative polymers, such as the polymers sold under the names Pemulen TR1 or TR2 by the company Goodrich, Salcare SC90 by the company Allied Colloids, Aculyn 22, 28, 33, 44 or 46 by the company Rohm & Haas and Elfacos T210 and T212 by the company Akzo or else sodium polyacrylate such as the product sold by the company Sensient under the commercial reference Covacryl MV 60, or else the polymer sold under the name Carbopol Ultrez 21 by the company Lubrizol.

The inorganic thickeners may essentially consist of inorganic oxides and/or hydroxides.

They may preferably be chosen from clays and silicas.

Clays are products that are already well known per se, which are described, for example, in the publication Minéralogie des argiles [Mineralogy of Clays], S. Caillère, S. Hénin, M. Rautureau, 2nd Edition 1982, Masson, the teaching of which is included herein by way of reference.

Clays are silicates containing a cation that may be chosen from calcium, magnesium, aluminium, sodium, potassium and lithium cations, and mixtures thereof.

Mention may be made, as examples of such products, of clays of the family of the smectites, such as laponites and montmorillonites, hectorites, bentonites, beidellites or saponites, and also of the family of the vermiculites, stevensite or chlorites.

The clays may be of natural or synthetic origin. Preferably, clays that are cosmetically compatible and acceptable with keratin fibres such as the hair are used.

The clay can be chosen from montmorillonite, bentonite, hectorite, attapulgite, sepiolite and mixtures thereof. Preferably, the clay is a bentonite or a hectorite.

The clays may be chosen from organophilic clays.

Organophilic clays are clays modified with a chemical compound chosen from quaternary amines, tertiary amines, amine acetates, imidazolines, amine soaps, fatty sulfates, alkyl aryl sulfonates and amine oxides, and mixtures thereof.

Preferably, the organophilic clays according to the invention are clays modified with a chemical compound chosen from quaternary amines.

Organophilic clays that may be mentioned include quaternium-18 bentonites such as those sold under the names Bentone 3, Bentone 38 and Bentone 38V by Elementis, Tixogel VP by United Catalyst, and Claytone 34, Claytone 40 and Claytone XL by Southern Clay; stearalkonium bentonites such as those sold under the names Bentone 27V by Elementis, Tixogel LG by United Catalyst, and Claytone AF and Claytone APA by Southern Clay; and quaternium-18/benzalkonium bentonites such as those sold under the names Claytone HT and Claytone PS by Southern Clay.

The organophilic clay is particularly chosen from modified hectorites such as the hectorite modified by C₁₀-C₁₂ fatty acid ammonium chloride, in particular distearyldimethylammonium chloride and stearylbenzyldimethylammonium chloride.

The silicas that can be used may be natural and untreated. Mention may thus be made of the silicas provided under the names Sillitin N85, Sillitin N87, Sillitin N82, Sillitin V85 and Sillitin V88 by Hoffmann Mineral.

They may be fumed silicas.

The fumed silicas can be obtained by high-temperature hydrolysis of a volatile silicon compound in an oxyhydrogen flame, producing a finely divided silica. This process makes it possible in particular to obtain hydrophilic silicas which contain a large number of silanol groups at their surface. It is possible to chemically modify the surface of said silica via a chemical reaction which brings about a reduction in the number of silanol groups. It is possible in particular to substitute silanol groups with hydrophobic groups; a hydrophobic silica is then obtained.

The hydrophobic groups can be:

(a) trimethylsiloxyl groups, which are obtained in particular by treating fumed silica in the presence of hexamethyldisilazane. Silicas thus treated are known as “Silica silylate” according to the CTFA (6th Edition, 1995);

(b) dimethylsilyloxyl or polydimethylsiloxane groups, which are obtained in particular by treating fumed silica in the presence of polydimethylsiloxane or dimethyldichlorosilane. Silicas thus treated are known as “Silica dimethyl silylate” according to the CTFA (6th Edition, 1995).

Functionalized silicas that may be mentioned include the products provided under the names Aktisil MAM, Aktisil MAM-R and Aktisil WW by the company Hoffmann Mineral.

Preferably, the inorganic thickener(s) is (are) chosen from laponites, montmorillonite, bentonite, hectorite, attapulgite, sepiolite, smectite, kaolin, silica derivatives and clays.

The thickener(s) may be present in a content ranging from 0.01% to 10% by weight, preferably in a content ranging from 0.1% to 5% by weight and more preferably from 0.2% to 2% by weight relative to the total weight of the composition.

The composition according to the invention may also comprise at least one surfactant.

The surfactants are chosen from anionic, cationic, non-ionic, amphoteric and zwitterionic surfactants.

The term “anionic surfactant” is intended to mean a surfactant comprising, as ionic or ionizable groups, only anionic groups. These anionic groups are preferably chosen from the groups —C(O)OH, —C(O)O⁻, —SO₃H, —S(O)₂O⁻, —OS(O)₂OH, —S(O)₂O⁻, —P(O)OH₂, —P(O)₂O⁻, —P(O)O₂⁻, —P(OH)₂, ═P(O)OH, —P(OH)O⁻, ═P(O)O⁻, ═POH and ═PO⁻, the anionic parts comprising a cationic counterion such as an alkali metal, an alkaline-earth metal or an ammonium.

By way of examples of anionic surfactants that may be used in the composition according to the invention, mention may be made of alkyl sulfates, alkyl ether sulfates, alkylamido ether sulfates, alkylarylpolyether sulfates, monoglyceride sulfates, alkylsulfonates, alkylamidesulfonates, alkylarylsulfonates, α-olefin sulfonates, paraffin sulfonates, alkylsulfosuccinates, alkylether sulfosuccinates, alkylamide sulfosuccinates, alkylsulfoacetates, acylsarcosinates, acylglutamates, alkylsulfosuccinamates, acylisethionates and N-acyltaurates, salts of alkyl monoesters of polyglycoside-polycarboxylic acids, acyllactylates, D-galactoside uronic acid salts, alkyl ether carboxylic acid salts, alkylaryl ether carboxylic acid salts, alkylamido ether carboxylic acid salts; and the corresponding non-salified forms of all these compounds; the alkyl and acyl groups of all these compounds comprising from 6 to 40 carbon atoms and the aryl group denoting a phenyl group.

These compounds can be oxyethylenated and then preferably comprise from 1 to 50 ethylene oxide units.

The salts of C₆-C₂₄ alkyl monoesters of polyglycoside-polycarboxylic acids can be chosen from C₆-C₂₄ alkyl polyglycoside-citrates, C₆-C₂₄ alkyl polyglycoside-tartrates and C₆-C₂₄ alkyl polyglycoside-sulfosuccinates.

When the anionic surfactant(s) are in salt form, it/they may be chosen from alkali metal salts such as the sodium or potassium salt and preferably the sodium salt, ammonium salts, amine salts and in particular amino alcohol salts or alkaline-earth metal salts such as the magnesium salts.

By way of examples of amino alcohol salts, mention may in particular be made of monoethanolamine, diethanolamine and triethanolamine salts, monoisopropanolamine, diisopropanolamine or triisopropanolamine salts, 2-amino-2-methyl-1-propanol salts, 2-amino-2-methyl-1,3-propanediol salts and tris(hydroxymethyl)aminomethane salts.

Alkali metal or alkaline-earth metal salts, and in particular sodium or magnesium salts, are preferably used.

Use is preferably made, among the anionic surfactants mentioned, of (C₆-C₂₄)alkyl sulfates, (C₆-C₂₄)alkyl ether sulfates comprising from 2 to 50 ethylene oxide units, in particular in the form of alkali metal, ammonium, amino alcohol and alkaline-earth metal salts, or a mixture of these compounds.

In particular, it is preferable to use (C₁₂-C₂₀)alkyl sulfates, (C₁₂-C₂₀)alkyl ether sulfates comprising from 2 to 20 ethylene oxide units, in particular in the form of alkali metal, ammonium, amino alcohol and alkaline-earth metal salts, or a mixture of these compounds. Better still, it is preferred to use sodium lauryl ether sulfate containing 2.2 mol of ethylene oxide.

Examples of non-ionic surfactants that may be used in the compositions of the present invention are described, for example, in the Handbook of Surfactants by M. R. Porter, published by Blackie & Son (Glasgow and London), 1991, pp 116-178. They are in particular chosen from alcohols, α-diols and (C₁-C₂₀)alkylphenols, these compounds being etherified with ethoxylated, propoxylated or glycerolated groups and containing at least one fatty chain comprising, for example, from 8 to 40 carbon atoms, the number of ethylene oxide and/or propylene oxide groups possibly ranging in particular from 1 to 200, and the number of glycerol groups possibly ranging in particular from 1 to 30.

Mention may also be made of condensates of ethylene oxide and of propylene oxide with fatty alcohols; polyethoxylated fatty amides preferably having from 2 to 30 ethylene oxide units, polyglycerolated fatty amides containing on average from 1 to 5 and in particular from 1.5 to 4 glycerol groups, ethoxylated fatty acid esters of sorbitan containing from 2 to 30 ethylene oxide units, fatty acid esters of sucrose, fatty acid esters of polyethylene glycol, (C₆-C₂₄ alkyl)polyglycosides, N—(C₆-C₂₄ alkyl)glucamine derivatives, amine oxides such as (C₁₀-C₁₄ alkyl)amine oxides or N—(C₁₀-C₁₄ acyl)aminopropylmorpholine oxides.

The amphoteric or zwitterionic surfactants which can be used in the present invention may in particular be secondary or tertiary aliphatic amine derivatives in which the aliphatic group is a linear or branched chain containing from 8 to 22 carbon atoms, said amine derivatives containing at least one anionic group, for instance a carboxylate, sulfonate, sulfate, phosphate or phosphonate group. Mention may be made in particular of (C₈-C₂₀ alkyl)betaines, sulfobetaines, (C₈-C₂₀ alkyl)amido(C₆-C₈ alkyl)betaines and (C₈-C₂₀ alkyl)amido(C₆-C₈ alkyl)sulfobetaines.

Among the optionally quaternized secondary or tertiary aliphatic amine derivatives that can be used, as defined above, mention may also be made of the compounds classified in the CTFA dictionary, 5th edition, 1993, under the names disodium cocoamphodiacetate, disodium lauroamphodiacetate, disodium caprylamphodiacetate, disodium capryloamphodiacetate, disodium cocoamphodipropionate, disodium lauroamphodipropionate, disodium caprylamphodipropionate, disodium capryloamphodipropionate, lauroamphodipropionic acid and cocoamphodipropionic acid.

By way of example, mention may be made of the cocoamphodiacetate sold by the company Rhodia under the trade name Miranol® C2M Concentrate.

Use may also be made of the compound classified in the CTFA dictionary under the name sodium diethylaminopropyl cocoaspartamide and sold by the company Chimex under the name Chimexane HB.

Among the amphoteric or zwitterionic surfactants mentioned above, use is preferably made of (C₈-C₂₀)alkylbetaines such as cocobetaine, (C₈-C₂₀)alkylamido(C₃-C₈)alkylbetaines such as cocamidopropylbetaine, the compounds of formula (B′2), such as the sodium salt of diethylaminopropyl laurylaminosuccinamate (INCI name: sodium diethylaminopropyl cocoaspartamide) and mixtures thereof. More preferentially, the amphoteric or zwitterionic surfactant(s) is (are) chosen from cocamidopropylbetaine and cocobetaine.

The cationic surfactant(s) that can be used in the composition according to the invention comprise(s), for example, optionally polyoxyalkylenated primary, secondary or tertiary fatty amine salts, quaternary ammonium salts, and mixtures thereof.

Examples of quaternary ammonium salts that may in particular be mentioned include:
tetraalkylammonium chlorides, such as, for example, dialkyldimethylammonium or alkyltrimethylammonium chlorides in which the alkyl group comprises approximately from 12 to 22 carbon atoms, in particular behenyltrimethylammonium, distearyldimethylammonium, cetyltrimethylammonium or benzyldimethylstearylammonium chlorides, or else, secondly, distearoylethylhydroxyethylmethylammonium methosulfate, dipalmitoyl-ethylhydroxyethylammonium methosulfate or distearoylethylhydroxyethylammonium methosulfate, or else, lastly, palmitylamidopropyltrimethylammonium chloride or stearamidopropyldimethyl(myristyl acetate)ammonium chloride, sold under the name Ceraphyl® 70 by the company Van Dyk;
quaternary ammonium salts of imidazoline, for instance the product sold under the name Rewoquat® W 75 by the company Rewo;
diquaternary or triquaternary ammonium salts, for instance Finquat CT-P, provided by the company Finetex (Quaternium 89), and Finquat CT, provided by the company Finetex (Quaternium 75);
quaternary ammonium salts containing one or more ester functions, such as the salts, in particular the chloride or methyl sulfate, of diacyloxyethyldimethylammonium, diacyloxyethylhydroxyethylmethylammonium, monoacyloxyethyldihydroxyethylmethylammonium, triacyloxyethylmethylammonium or monoacyloxyethylhydroxyethyldimethylammonium, and mixtures thereof. The acyl groups preferably contain 14 to 18 carbon atoms and are obtained more particularly from a vegetable oil, such as palm oil or sunflower oil. When the compound contains several acyl groups, these groups may be identical or different.

These products are obtained, for example, by direct esterification of triethanolamine, triisopropanolamine, an alkyldiethanolamine or an alkyldiisopropanolamine, which are optionally oxyalkylenated, with fatty acids or with mixtures of fatty acids of vegetable or animal origin, or by transesterification of their methyl esters. This esterification is followed by a quaternization by means of an alkylating agent, such as an alkyl halide, preferably methyl or ethyl halide, a dialkyl sulfate, preferably dimethyl or diethyl sulfate, methyl methanesulfonate, methyl para-toluenesulfonate, glycol chlorohydrin or glycerol chlorohydrin.

Such compounds are sold, for example, under the names Dehyquart® by the company Henkel, Stepanquat® by the company Stepan, Noxamium® by the company Ceca or Rewoquat® WE 18 by the company Rewo-Witco.

The composition according to the invention may contain, for example, a mixture of quaternary ammonium salts of monoesters, diesters and triesters with a weight majority of diester salts.

It is also possible to use the ammonium salts containing at least one ester function that are described in patents U.S. Pat. No. 4,874,554 and U.S. Pat. No. 4,137,180.

Use may be made of behenoylhydroxypropyltrimethylammonium chloride, sold by Kao under the name Quatarmin BTC 131.

Preferably, the ammonium salts containing at least one ester function contain two ester functions.

Among the cationic surfactants that may be present in the composition according to the invention, it is more particularly preferred to choose cetyltrimethylammonium, behenyltrimethylammonium and dipalmitoylethylhydroxyethylmethylammonium salts, and mixtures thereof, and more particularly behenyltrimethylammonium chloride, cetyltrimethylammonium chloride and dipalmitoylethylhydroxyethylammonium methosulfate, and mixtures thereof.

When they are present, the surfactants may represent from 0.01% to 20%, preferably from 0.1% to 10% by weight and better still from 0.5% to 2% by weight, relative to the total weight of the composition.

The pH of the composition according to the invention generally ranges from 2 to 9 and in particular from 3 to 8. It may be adjusted to the desired value by means of acidifying or basifying agents normally used in the cosmetics industry for this type of application, or alternatively using standard buffer systems.

Among the acidifying agents, mention may be made, by way of example, of organic acids or inorganic acids.

The term “inorganic acid” is intended to mean any acid derived from an inorganic compound. Among the inorganic acids, mention may be made of hydrochloric acid, orthophosphoric acid, sulfuric acid, sulfonic acids and nitric acid.

Use may be made in particular of inorganic or organic acids such as hydrochloric acid, orthophosphoric acid or sulfuric acid, carboxylic acids, for instance acetic acid, tartaric acid, citric acid or lactic acid, and sulfonic acids.

Among the basifying agents, examples that may be mentioned include aqueous ammonia, alkali metal carbonates, alkanolamines, such as mono-, di- and triethanolamines and derivatives thereof, sodium hydroxide, potassium hydroxide and the compounds of the following formula:

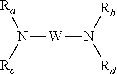

in which W is a propylene residue optionally substituted with a hydroxyl group or a C₁-C₄ alkyl group; Rₐ, R_{b}, R_{c} and R_{d}, which may be identical or different, represent a hydrogen atom or a C₁-C₄ alkyl or C₁-C₄ hydroxyalkyl group.

Preferably, the pH modifiers may be chosen from alkaline agents such as aqueous ammonia, monoethanolamine, diethanolamine, triethanolamine, 1,3-propanediamine or an alkaline hydroxide, such as 2-amino-2-methyl-1-propanol, or else acidifying agents, such as phosphoric acid or hydrochloric acid.

The composition according to the invention may comprise a propellant. For example, mention may be made of liquefied gases such as dimethyl ether, 1,1-difluoroethane, or C₃₋₅ alkanes, for instance propane, isopropane, n-butane, isobutane or pentane, or compressed gases such as air, nitrogen or carbon dioxide, and mixtures thereof.

Mention may be made preferentially of C₃₋₅ alkanes and in particular propane, n-butane and isobutane, and mixtures thereof.

When it comprises propellant(s), the composition comprises one or more propellant(s) in an amount ranging from 1% to 60% by weight, better still from 2% to 50% by weight and even more preferentially from 4% to 40% by weight relative to the total weight of the composition.

The composition used in the process according to the invention may also contain one or more additives other than the compounds of the invention, chosen from non-silicone conditioning agents and in particular cationic polymers, vitamins and provitamins including panthenol, sunscreens, nacreous agents and opacifiers, dyes, sequestrants, plasticizers, solubilizers, acidifying agents, basifying agents, antioxidants, antifoams, moisturizers, emollients, hydroxy acids, penetrants, fragrances, preserving agents and fillers and particles other than the insoluble particles comprising a material capable of changing state, for instance coloured or colourless inorganic and organic pigments.

These additives may be present in the composition according to the invention in an amount ranging from 0 to 20% by weight relative to the total weight of the composition.

Needless to say, those skilled in the art will take care to select the optional additional compounds and/or the amount thereof such that the advantageous properties of the compositions used according to the invention are not, or are not substantially, adversely affected by the envisaged addition.

The composition according to the invention may be, inter alia, in the form of liquids that are thickened to a greater or lesser degree, lotions, gels, sera, creams, pastes or mousses.

Preferably, the composition according to the invention is in the form of gels or mousses.

The composition may be packaged in a container which does not have dispensing means such as a pump or a valve, for example in a bottle, a tube or a jar.

The process for cosmetic treatment of the hair according to the invention consists in applying to the hair an effective amount of a composition as described above.

The composition can be applied to wet or dry hair.

The composition can be applied to the entire head of hair or locally, to just one part of the head of hair.

The hair can be shaped with the hands or using a comb or a brush.

After or simultaneously with the shaping, one or more stimuli enabling the change of state of the material is (are) applied.

The application of the stimulus can be carried out directly after having distributed the composition on the head of hair. The stimulus can be applied gradually to the entire region of the head of hair on which the composition was applied.

The stimulus is applied for a period of time sufficient for the particles to change state. Preferably, the stimulus enabling the change of state of the material is applied for a period of time ranging from 30 seconds to 3 hours, preferably ranging from 1 to 20 minutes.

The stimulus for obtaining fixing of the hairstyle is then stopped.

Through successive applications of the stimulus, the hairstyle can be custom-remodelled as many times as desired without further provision of composition.

Preferably, the hair shaping process according to the invention does not comprise a step during which a fixing polymer is applied.

The examples which follow serve to illustrate the invention without, however, being limiting in nature.

### EXAMPLES

The compositions used in the process according to the invention were prepared from the compounds indicated in the table below. In the following examples, all the amounts are shown as percentage by weight of active materials, relative to the total weight of the composition.

### Compositions in Gel Form:

| | | 1 | 2 | 3 | 4 | 5 | 6 | 7 | 8 | 9 |
|---|---|---|---|---|---|---|---|---|---|---|
| Particles | Polypropylene ⁽¹⁾ | 5 | 5 | 5 | 2.5 | 1.5 | 1.5 | 3.5 | 2.5 | 1.5 |
| | Bis(trimethylolpropane) | | | | | 3.5 | 3.5 | 1.5 | 2.5 | 3.5 |
| | tetrastearate ⁽²⁾ | | | | | | | | | |
| | Polyvinyloctadecyl | | | | 2.5 | | | | | |
| | ether ⁽³⁾ | | | | | | | | | |
| | Ethanol | | 10 | | | | 10 | 2 | 2 | 2 |
| | Laponite ⁽⁴⁾ | | | 0.5 | | | | | | 0.2 |
| | Acrylates/C10-30 alkyl | 0.24 | 0.25 | 0.25 | 0.25 | 0.25 | 0.25 | 0.25 | 0.25 | 0.20 |
| | acrylate crosspolymer⁽⁵⁾ | | | | | | | | | |
| | 2-amino-2-methylpropanol | qs pH 7.5 | qs pH 7.5 | qs pH 7.5 | qs pH 7.5 | qs pH 7.5 | qs pH 7.5 | qs pH 7.5 | qs pH 7.5 | qs pH 7.5 |
| | Water | qs 100 | qs 100 | qs 100 | qs 100 | qs 100 | qs 100 | qs 100 | qs 100 | qs 100 |

⁽¹⁾ Licocare PP202 LP 3333 sold by the company Clariant
⁽²⁾ Hest 2T-4S sold by the company Heterene
⁽³⁾ Giovarez 1800 sold by the company Phoenix Chemical
⁽⁴⁾ Laponite XLG sold by the company Rockwood Additives
⁽⁵⁾Carbopol Ultrez 21 sold by the company Lubrizol

### Compositions in Mousse Form

| | | 10 | 11 | 12 |
|---|---|---|---|---|
| | Isobutane/butane/propane (56/24/20)⁽¹⁾ | 5 | 5 | 5 |
| Particles | Polypropylene⁽²⁾ | 1.425 | 2.85 | 2.375 |
| | Bis(trimethylolpropane) tetrastearate⁽³⁾ | 3.325 | 6.65 | 2.375 |
| | Hexamethyl diisocianate/polyethylene glycol | 0.95 | 0.95 | 0.95 |
| | polyoxyethylenated copolymer containing alpha-omega | | | |
| | stearyl end groups⁽⁴⁾ | | | |
| | Glycerol | 4.75 | 4.75 | 4.75 |
| | Propylene glycol | 2.375 | 2.375 | 2.375 |
| | Oxyethylenated lauryl alcohol (4 OE)⁽⁵⁾ | 0.95 | 0.95 | 0.95 |
| | preserving agents | 1.045 | 1.045 | 1.045 |
| | Water | qs | qs | qs |
| | | 100 | 100 | 100 |

⁽¹⁾Drivosol 32 A sold by the company Evonik Oxeno
⁽²⁾Licocare PP202 LP 3333 sold by the company Clariant
⁽³⁾Hest 2T-4S sold by the company Heterene
⁽⁴⁾Rheolate FX 1100 sold by the company Elementis
⁽⁵⁾Brij L4-LQ-(SG) sold by the company Croda

The melting point of the materials and of the mixtures of materials forming the particles described in the table above are the following:*

Polypropylene⁽¹⁾: 71° C. (Examples 1, 2 and 3)

Bis(trimethylolpropane) tetrastearate⁽²⁾: 35° C.

Polyvinyloctadecyl ether⁽³⁾: 42-48° C.

Polypropylène⁽¹⁾/Polyvinyloctadecyl ether⁽³⁾ mixture (50/50): 63-65° C. (Example 4)

Polypropylène⁽¹⁾/Bis(trimethylolpropane) tetrastearate⁽²⁾ mixture (30/70): 48° C. (Examples 5, 6, 9, 10 and 11)

Polypropylène⁽¹⁾/Bis(trimethylolpropane) tetrastearate⁽²⁾ mixture (70/30): 48° C. Example 7

Polypropylène⁽¹⁾/Bis(trimethylolpropane) tetrastearate⁽²⁾ mixture (50/50): 66° C. (Examples 8 and 12).

The compositions described in the various examples are packaged in jars.

They are sampled and applied by hand to dry or wet hair.

The hairstyle is then shaped and heat is provided by a conventional hairdryer blowing at a temperature of 60° C. for 3 to 5 minutes per half a head so as to melt the particles. The hairdryer is applied gently to the entire surface of the head of hair so that the particles melt.

Once the particles have melted, the hairdryer is removed from the head of hair and the particles become invisible on the head of hair which is fixed via spots forming a network of hair comparable to that obtained with a lacquer.

The hair shaping is heat-reversible, it being possible to give the lock of hair a new shape if it is again subjected to heat.

A very good long-lasting sheen and a very good long-lasting feel of the hair are also obtained.

1. Process for cosmetic treatment of the hair, in particular hair shaping process, comprising the following steps:
applying to the hair a composition comprising a liquid solvent system consisting of one or more solvents and a dispersion of particles that are insoluble in the solvent system, the particles comprising a material capable of changing state under the effect of an external stimulus,
before, during or after the applying of the composition, shaping the hair to the desired hairstyle, then or simultaneously,
applying one or more stimuli enabling the change of state of the material,
stopping the stimulus or stimuli for obtaining fixing of the hairstyle.

2. Process according to claim 1, in which the stimulus enabling the change of state of the material is chosen from heat, mechanical, pH-variation and light stimuli, and is preferably a heat stimulus.

3. Process according to the preceding claim, in which the heat stimulus is applied by means of a hairdryer, a heating hood, infrared radiation or microwaves.

4. Process according to any one of the preceding claims, in which the material is chosen from thermoplastic polymers, waxes, pasty fatty substances or mixtures thereof.

5. Process according to the preceding claim, in which the thermoplastic polymers are chosen from polyolefins such as polyethylenes and polypropylenes, polyamides, polyethers, polyurethanes or copolymers of these compounds.

6. Process according to either one of claims 4 and 5, in which the waxes are chosen from waxes of natural origin, preferably chosen from waxes of animal, vegetable or mineral origin, and the synthetic waxes are preferably chosen from nonpolar waxes and ester waxes, and preferably from waxes of polypropylene and of bis(trimethylolpropane) tetrastearate.

7. Process according to any one of the preceding claims, in which the material has a melting/softening point ranging from 30° C. to 150° C., preferably from 30° C. to 120° C., even more preferentially from 35° C. to 80° C.

8. Process according to the preceding claim, in which the solvent(s) has (have) a boiling point above the melting/softening point of the material.

9. Process according to any one of the preceding claims, in which the solvent(s) is (are) chosen from water and organic solvents, preferably chosen from monoalcohols, polyols and polyol ethers, and mixtures thereof.

10. Process according to any one of the preceding claims, in which the solvent(s) is (are) chosen from water, ethanol or mixtures thereof.

11. Process according to any one of the preceding claims, in which the particles have a number-average primary size ranging from 0.01 to 500 μm, preferably ranging from 0.1 to 300 μm and more preferentially from 1 to 250 μm.

12. Process according to any one of the previous claims, in which the particles are present in proportions of at least 0.05% by weight, preferably ranging from 0.1% to 95% by weight, more preferentially from 1% to 50% by weight and better still from 2% to 30% by weight relative to the total weight of the composition.

13. Process according to any one of the preceding claims, in which the composition comprises a thickener, preferably an inorganic thickener.

14. Process according to any one of the preceding claims, in which the stimulus or stimuli enabling the change of state of the material is (are) applied for a period of time ranging from 30 seconds to 3 hours, preferably ranging from 1 to 15 minutes.

15. Process according to any one of the preceding claims, in which the material is capable of changing state going from a solid state to a liquid state.

16. Process according to any one of the preceding claims, in which the material is capable of reversibly changing state.

17. Process according to any one of the preceding claims, in which the composition is in the form of a gel, a lotion or a mousse.

18. Process according to any one of the preceding claims, which does not comprise a step in which a fixing polymer is applied.
